(19) United States
(12) Patent Application Publication
(10) Pub. No.: US 2013/0217024 A1
(43) Pub. Date: Aug. 22, 2013

(54) METHODS FOR DIAGNOSING BACTEREMIA AND FUNGEMI

(71) Applicant: Gary W. Procop, Twinsburg, OH (US)

(72) Inventor: Gary W. Procop, Twinsburg, OH (US)

(73) Assignee: THE CLEVELAND CLINIC FOUNDATION, Cleveland, OH (US)

(21) Appl. No.: 13/771,594

(22) Filed: Feb. 20, 2013

Related U.S. Application Data
(60) Provisional application No. 61/600,797, filed on Feb. 20, 2012.

(51) Int. Cl. C12Q 1/68

(52) U.S. Cl. CPC C12Q 1/689; C12Q 1/6895
USPC 435/6.12; 435/6.15

(57) ABSTRACT

A method for diagnosing bacteremia and/or fungemia can include the steps of obtaining a blood sample from the subject, contacting the blood sample with a lysing agent under conditions in which both red and white blood cells are lysed in the blood sample and bacterial and/or fungal cells remain intact, extracting bacterial and/or fungal nucleic acids from bacterial and/or fungal cells in the blood sample (respectively), and detecting the presence of bacterial and/or fungal nucleic acids in the blood sample, wherein the presence of bacterial and/or fungal nucleic acids in the blood sample is indicative of the subject having bacteremia and/or fungemia, respectively.

### RELATED APPLICATION

This application claims the benefit of U.S. Provisional Patent Application Ser. No. 61/600,797, filed Feb. 20, 2012, the entirety of which is hereby incorporated by reference for all purposes.

### TECHNICAL FIELD

The present disclosure relates generally to diagnostic methods, and more particularly to methods for diagnosing bacteremia and fungemia.

### BACKGROUND

Disseminated tuberculosis (TB) is a contagious bacterial infection that has spread from the lungs to other parts of the body through the blood or lymph system. Disseminated tuberculosis is a major health problem in countries where generalized HIV epidemics coincide with high tuberculosis incidence rates, often causing fatal illness in patients with immunologically advanced HIV diseases. Mycobacterium tuberculosis, first recognized as a cause of bloodstream infection almost a century ago, is today a leading cause of community-acquired bloodstream infection among febrile hospitalized patients in sub-Saharan Africa and Asia.

In-hospital case fatality rates for bacteremic disseminated tuberculosis in the era before the widespread availability of antiretroviral therapy approached 50%. Since the median survival of patients with bacteremic disseminated tuberculosis following admission hospital may be less than one week, early recognition and treatment can be important to avert mortality.

An important limitation of mycobacterial blood culture for the diagnosis of disseminated tuberculosis is that the mean time to a positive result exceeds the median survival of patients with the disease in endemic areas. Mycobacterial blood culture methods, such as those using visual inspection of processed blood inoculated on a solid medium (e.g., the ISOLATOR 10 system) and continuous detection in liquid medium inoculated with blood (e.g., the BACTEC MYCO/F LYTIC or BacT/ALERT MB system) provide a means to support the laboratory diagnosis of disseminated tuberculosis.

Despite using blood culture systems with continuous detection, the time to positive may be too long to affect clinical decision making in the majority of instances. For example, in a United States evaluation of the performance of the BACTEC 13A, MYCO/F LYTIC, BacT/ALERT MB, and ISOLATOR 10 systems for detection of mycobacteremia, it was found that bottles yielding M. tuberculosis signaled positive after a mean of 22.8 to 28.0 days incubation compared with 9.9 to 20.4 days for bottles with MAC in the systems studied. In a Tanzanian study, MB bottles signaled positive for M. tuberculosis after a mean (range) incubation time of 22.6 (9.4, 37.5) days in the BacT/ALERT MB system. Consequently, the diagnosis is often overlooked and is a frequent finding at post-mortem examination of HIV-infected patients in areas with high rates of tuberculosis.

While increased awareness among clinicians is important to identify patients with disseminated tuberculosis, its clinical manifestations may be non-specific, and there remains a need for new approaches for the rapid laboratory diagnosis of the bacteremic disease. In North America, the incidence of tuberculosis has declined. Bacteremia and fungemia, however, remain an important cause of disease, particularly nosocomial infections. The detection of agents responsible for bacteremia and fungemia also require the use of culture, which is time-consuming and costly.

### SUMMARY

The present application relates to methods for the diagnosis of bacteremia and/or fungemia in a subject. In one aspect of the present disclosure, a method for diagnosing bacteremia and/or fungemia in a subject can include the steps of: obtaining a blood sample from the subject; contacting the blood sample with a lysing agent under conditions in which both red and white blood cells are lysed in the blood sample and bacterial and/or fungal cells remain intact; removing the lysate (which contains DNA, e.g., human DNA); extracting bacterial and/or fungal nucleic acids from bacterial and/or fungal cells in the blood sample, respectively; and detecting the presence of bacterial and/or fungal nucleic acids in the blood sample. The presence of bacterial and/or fungal nucleic acids in the blood sample may be indicative of the subject having bacteremia and/or fungemia, respectively.

In some instances, the lysing agent can include a saponin. In other instances, the bacteremia diagnosed can be Mycobacterium tuberculosis bacteremia. In further instances, the volume of the blood sample obtained from a subject can be about 1 ml to about 40 ml (e.g., about 4 ml to 6 ml). In one example, the blood sample obtained from a subject can be whole blood.

In some instances, the step of extracting bacterial and/or fungal nucleic acids from the blood sample can include: contacting the blood sample with a bacterial and/or fungal lysing agent to release the bacterial and/or fungal nucleic acids sought to be detected; and isolating the bacterial and/or fungal nucleic acids. In other instances, the step of detecting the presence of bacterial and/or fungal nucleic acids from the blood sample can include post-amplification melt-curve analysis.

A method of diagnosing bacteremia and/or fungemia in a subject can further include the step of amplifying at least one segment of the bacterial nucleic acids in the blood sample. This step can include RT-PCR targeting of at least one segment of a bacterial gene for mycobacterial 16S rRNA.

In another aspect of the present disclosure, a method for diagnosing Mycobacterium tuberculosis bacteremia can include the steps of: obtaining a blood sample from the subject; contacting the blood sample with a lysing agent under conditions in which both red and white blood cells are lysed in the blood sample and Mycobacterium tuberculosis cells remain intact; removing the lysate (which contains DNA, e.g., human DNA); extracting nucleic acids from Mycobacterium tuberculosis cells in the blood sample; and detecting the presence of Mycobacterium tuberculosis nucleic acids in the blood sample. The presence of mycobacterium tuberculosis nucleic acids in the blood sample may be indicative of the subject having Mycobacterium tuberculosis bacteremia.

In some instances, the lysing agent can include a saponin. In other instances, the bacteremia diagnosed can be Mycobacterium tuberculosis bacteremia. In further instances, the volume of the blood sample obtained from a subject can be about 1 ml to about 40 ml (e.g., about 4 ml to 6 ml). In one example, the blood sample obtained from a subject can be whole blood.

In other instances, the step of extracting mycobacterium tuberculosis nucleic acids from the blood sample can include: contacting the blood sample with a bacterial lysing agent to release the Mycobacterium tuberculosis nucleic acids sought to be detected; and isolating the Mycobacterium tuberculosis nucleic acids. In other instances, the step of detecting the presence of Mycobacterium tuberculosis nucleic acids from the blood sample can include post-amplification melt-curve analysis.

A method of diagnosing Mycobacterium tuberculosis bacteremia in a subject can further include the step of amplifying at least one portion of the Mycobacterium tuberculosis nucleic acids in the blood sample. This step can include RT-PCR targeting of at least one portion of a Mycobacterium tuberculosis gene that encodes a mycobacterial 16S rRNA.

Another aspect of the present disclosure can include a kit for diagnosing bacteremia and/or fungemia in a subject.

### DESCRIPTION OF DRAWINGS

The foregoing and other features of the present disclosure will become apparent to those skilled in the art to which the present disclosure relates upon reading the following description with reference to the accompanying drawings, in which:

FIG. 1 is a flowchart illustrating a method for diagnosing bacteremia and/or fungemia in a subject according to one aspect of the present disclosure.

### DETAILED DESCRIPTION

### Definitions

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the present invention.

In the context of the present disclosure, the term “subject” can refer to any animal, including, but not limited to, humans and non-human animals (e.g., rodents, arthropods, insects, fish (e.g., zebrafish)), non-human primates, ovines, bovines, ruminants, lagomorphs, porcines, caprines, equines, canines, felines, ayes, etc.). The terms “patient” and “subject” can be used interchangeably herein in reference to a human subject. In some instances, a subject is suspected of having, or is susceptible to, bacteremia and/or fungemia.

As used herein, the term “bacteremia” can refer to the presence of bacteria (e.g., viable bacteria) in the blood or organs of a subject. In one example, “bacteremia caused by Mycobacterium tuberculosis” or “Mycobacterium tuberculosis bacteremia” can refer to bacteremia in which at least some of the bacteria in the blood or organs are Mycobacterium tuberculosis. Other species of bacteria also may be present and/or causative of the bacteremia. Non-limiting examples of bacteria that may also be present and/or causative of bacteremia can include Staphylococcus aureus (S. aureus), a Streptococcus, Escherichia coli (E. coli), Pseudomonas aeruginosa (P. aeruginosa), Acinetobacter baumannii (A. baumannii), Enterococcus faecium (E. faecium), Enterococcus faecalis (E. faecalis), Enterobacter aerogenes (E. aerogenes), Enterobacter cloacae (E. cloacae), Clostridium difficile (C. difficile), Klebsiella pneumoniae (K. pneumoniae), a Salmonella, Bacillus anthracis, Listeria monocytogenes, Chlamydophila pneumoniae, Ureaplasma urealyticum, Mycoplasma hominis, Mycoplasma pneumoniae, Haemophilus influenzae and/or Campylobacter jejuni.

As used herein, the term “fungemia” can refer to the presence of fungi or yeast (e.g., viable fungi or yeast) in the blood or organs of a subject. Non-limiting examples of fungi that may be causative of fungemia can include species of Candida, Saccharomyces, Aspergillus and Cryptococcus.

As used herein, the term “diagnosis” can refer to a process aimed at determining if an individual or subject is afflicted with a disease or ailment. In some instances, the phrase “diagnosis of bacteremia and/or fungemia” can refer to a process aimed at one or more of: determining if a subject is likely to develop a bacteremia and/or fungemia; and determining if a subject is afflicted with a bacteremia and/or fungemia.

As used herein, the terms “nucleic acid” and “polynucleotide” can be used interchangeably. The terms can refer to a deoxyribonucleotide or ribonucleotide polymer in either single- or double-stranded form and, unless otherwise stated, can encompass known analogs of natural nucleotides that can function in a similar manner as naturally occurring nucleotides. The terms can also encompass nucleic acid-like structures with synthetic backbones, as well as amplification products.

As used herein, the term “hybridizing” can refer to the binding of two single-stranded nucleic acids via complementary base pairing. The term “specific hybridization” as used herein can refer to a process in which a nucleic acid molecule preferentially binds, duplexes, or hybridizes to a particular nucleic acid sequence under stringent conditions (e.g., in the presence of competitor nucleic acids with a lower degree of complementarity to the hybridizing strand). In some instances, the terms can refer to a process in which a nucleic acid fragment (or segment) sought to be detected from a test sample preferentially binds to a particular probe.

As used herein, the term “probe” can refer to a nucleic acid molecule of known sequence, such as a short DNA sequence (i.e., an oligonucleotide), a PCR product, or an mRNA isolate. Probes can be specific nucleic acid sequences to which nucleic acid fragments from a test sample can be hybridized. Probes can specifically bind to nucleic acids of complementary or substantially complementary sequence through one or more types of chemical bonds, such as through hydrogen bond formation.

As used herein, the term “and/or” can include any and all combinations of one or more of the associated listed items.

Overview

The present disclosure generally relates to methods and kits for diagnosing bacteremia and/or fungemia in a subject through the extraction and detection of bacterial and/or fungal nucleic acids in a blood sample obtained from the subject. The present disclosure is based, at least in part, on the discovery that bacterial nucleic acids can be amplified directly from large volume whole blood samples. In accordance with the present disclosure, the bacterial nucleic acids are not extracted directly from the whole blood; rather, there is firstly a separation of the bacterial cells from blood cellular DNA to improve the sensitivity and specificity of the detection. In other words, if only very few bacterial cells are present in a whole blood sample, the very small quantity of bacterial nucleic acids extracted from the blood sample can be detected against the background of cellular DNA (which is typically present in a very high concentration) so that the separation mentioned above leads to great advantages in terms of sensitivity. This discovery allows for the early identification of bacterial and/or fungal infected blood samples, which can be indicative of a positive diagnosis of bacteremia and/or fungemia in a subject.

Methods

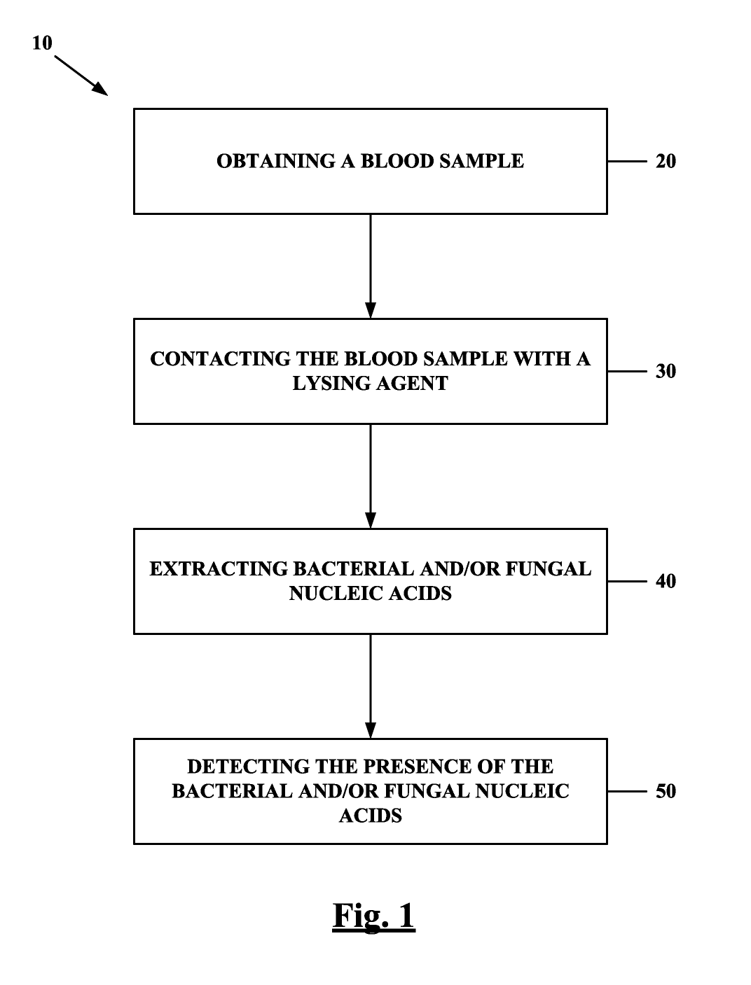

One aspect of the present disclosure can include a method 10 (FIG. 1) for diagnosing bacteremia and/or fungemia in a subject. Bacteremia and/or fungemia diagnosed in accordance with the present disclosure can include any bacteremia and/or fungemia resulting from bacteria and/or fungi, respectively, entering the bloodstream as a result of, for example, a severe complication of an infection (e.g., tuberculosis, pneumonia or meningitis), surgery (e.g., involving mucous membranes, such as the gastrointestinal tract), and catheters and other foreign bodies entering the vasculature (e.g., intravenous drug abuse). In one example, bacteremia can be caused by, or associated with, Mycobacterium tuberculosis.

In another aspect, the method 10 can include obtaining a blood sample from a subject (Step 20). In one example, the blood sample can be whole blood obtained from a human subject suspected of having, or at risk of developing, bacteremia and/or fungemia. A blood sample may be obtained, for example, using a hypodermic needle and syringe. In some instances, the volume of the blood sample obtained from the subject can be about 1 ml to about 40 ml. In other instances, the volume of the blood sample obtained from the subject can be about 4 ml to about 6 ml (e.g., 5 ml). In some instances, a whole blood sample can be treated with an anti-coagulant (e.g., EDTA or heparin) so that the whole blood sample does not coagulate too quickly. In some instances, the blood sample can be transferred to one or more containers (e.g., cryovials), frozen, and stored at −80° C. for later analysis in accordance with the method 10.

In further instances, Step 20 can include performing more than one blood draw so that lysates from multiple blood samples can be combined for use in the method 10. In such instances, the sensitivity of the method 10 can be increased as the concentration of bacteria and/or fungi available for analysis is increased with multiple blood draws. One skilled in the art will appreciate that obtained blood sample can be diluted about 2, 3, 4, 5, 10-fold or more. Additionally, it will be appreciated that other blood fractions, or any other fluids containing blood cells, may also be obtained at Step 20.

In another aspect, the blood sample can be contacted with a lysing agent for a time and under conditions sufficient to lyse blood cells (e.g., red and white blood cells) and thereby release any bacterial and/or fungal cells from, or associated with, the lysed blood cells (Step 30). In some instances, Step 30 can cause release of any phagocytosed bacterial and/or fungal cells so that very small quantities of bacterial and/or fungal cells can still be isolated and detected according to the method 10. Advantageously, it is no longer necessary that bacteria and/or fungi be freely dissolved in whole blood to enable their detection; rather, it is sufficient if a few bacterial and/or fungal cells are present in a blood sample after phagocytosis (e.g., in granulocytes or macrophages).

In some instances, a blood sample can be contacted with a lysing agent by collecting the blood sample directly into an evacuated collection tube that contains the lysing agent. In other instances, all or only a portion of a blood sample can be obtained from a subject and stored in a collection device (e.g., a collection tube), which can then be transferred to a separate collection device containing a lysing agent. Alternatively, the lysing agent can be added to a collection device after a blood sample has been transferred to the device.

Lysing agents can include any hemolytic surfactant, detergent, or other emulsifying agent. A review of various types of lysing agents is found in Thelestam & Möllby, 557 Biochim. Biophys. Acta 156 (1979). In some instances, the lysing agent can be a surfactant, such as a saponin that capable of lysing both red and white blood cells in a single step without lysing of bacterial and/or fungal cells (if present). The term “saponin”, as used herein, can include glycosidic triterpenoid compounds, which produce foam in aqueous solution, have hemolytic activity in most cases, and possess immune adjuvant activity. Saponins are classified as steroid or triterpenoid saponins, depending on the nature of the aglycone. The present disclosure includes the use of saponins per se, natural and pharmaceutically acceptable salts and pharmaceutically acceptable derivatives, and biologically active fragments thereof.

For a general review of saponins see, e.g.: Vogel, 11 Planta Med., 362 (1963); Tschesche and Wulff, 12 Planta Med., 272 (1964); Kochetkov and Khorlin, 16 Arzneim, Forsch 101 (1966); Kawasaki, 16 Sogo Rinsyo 1053 (1967); Tschesche, Kagakuno Ryoiki 571 (1971); Kawasaki, 11 Method. Chim. 87 (178); and Tschesche and Wulff, Fortschritee der Chemie Organischer Naturstoffe 461 (1973); Elks, 11 E Rodd's Chemistry of Carbon Compounds, 1 (1971); and Elks, 2D Rodd's Chemistry of Carbon Compounds, 205 (1974). For a discussion of triterpenoid saponins, see, e.g.: Basu and Rastogi, 6 Phytochemistry 1249 (1967); Agarwai and Rastogi, 13 Phytochemistry 2623 (1974); and Chandel and Rastogi, 19 Phytochemistry 1889 (1980). A third group of saponins, which contain nitrogen analogs of steroid sapongenins as aglycones, are called basic steroid saponins (see, e.g., Schreiber, 10 Alkaloids 1 (1968), Roddick, 13 Phytochemistry, 9 (1974), Herbert, 5 Alkaloids, 256 (1975), and Harrison, 6 Alkaloids, 285 (1976)).

Other lysing agents that may be employed in the present disclosure can include cationic surfactants (e.g., cetrimide and cetyl trimethylammonium bromide), detergents with similar structures, short-chain phospholipids, macrolides, various bacterial hemolysins, and certain snake venoms and toxins, such as toxic fractions from Gymnodium. It will be appreciated that blood cells may be lysed by any desired method including, for example, the use of a strong alkali, an enzymatic agent, osmotic shock, chaotropic concentrations of solutes, and/or sonic disruption.

After contacting the blood sample with a lysing agent (e.g., a hemolytic agent), bacterial and/or fungal cells remaining in the solution can be separated or isolated by centrifugation, for example, such that intact bacterial and/or fungal cells are separated from lysed red and white blood cells and are pelleted so that they can be resuspended in an appropriate buffer or media solution. While not necessary, it may be desirable to perform an additional step to wash and further purify the pelleted bacterial and/or fungal cells using an additional centrifugation procedure, for example. Since the solution used to wash the bacterial and/or fungal cells may also include a hemolytic agent, it is not necessary to completely lyse all of the red and/or white blood cells in the initial treatment. The amount of lysed red and/or white blood cells, however, should be sufficient to enable further analysis of the recovered bacterial and/or fungal cells.

In one example, whole blood can be aseptically collected in a 10 ml Wampole ISOLATOR tube (Alere, Inc., Waltham, Mass.) including a saponin. The tube can contain a concentration of a saponin that, when contacted with the blood sample for a sufficient period of time is effective in lysing both red and white blood cells in one step, thereby releasing a majority of intracellular bacteria and/or fungi (e.g., mycobacteria) therefrom. The collection tube can also include a foam retardant (e.g., polypropylene glycol), an anticoagulant (e.g., sodium polyanethol sulfate and/or EDTA), and/or a liquid plastic immiscible with water to concentrate bacteria and/or fungi (e.g., FLUORINERT, 3M, St. Paul, Minn.). Collection tubes can then be centrifuged to isolate and concentrate the bacterial and/or fungal cells in the sediment. Next, the supernatant can be discarded and the sediment (about 1.5 ml) added to an appropriate buffered solution (e.g., PBS) for recovery of bacteria and/or fungi. Alternatively, an appropriate bacterial and/or fungal recovery media solution can be used (e.g., Lowenstein-Jensen, Middlebrook 7H10, 7H11 or 7H11 selective, or BACTEC 13A).

Once the red and white blood cells in the blood sample are lysed, bacterial and/or fungal nucleic acids can be extracted at Step 40. In some instances, bacterial and/or fungal nucleic acids can be extracted by: contacting bacterial and/or fungal cells isolated from a blood sample lysate with a bacterial and/or fungal lysing agent to release the bacterial and/or fungal nucleic acids sought to be detected; and further separating or isolating the released bacterial and/or fungal nucleic acids from the solution. In one example, the bacterial and/or fungal nucleic acids can include bacterial and/or fungal DNA. In another example, the bacterial nucleic acids can include mycobacterial DNA. It will be appreciated that nucleic acid extraction procedures can be conducted to remove blood-associated PCR inhibitors.

Bacterial and/or fungal nucleic acids can be extracted from bacterial and/or fungal cells by, for example, contacting isolated bacterial and/or fungal cells with an appropriate lysis buffer and/or Proteinase K to digest protein and remove contamination from preparations. In one example, an appropriate mycobacteria lysis buffer can include tris-hydrochloride buffer (e.g., about 1-5% by weight), sodium lauroylsarcosine ionic surfactant (e.g., about 1-5% weight), and citric acid trisodium salt (e.g., about 10-20% by weight).

Bacterial and/or fungal nucleic acids can be extracted and further isolated from bacterial and/or fungal cells using a bench top automated nucleic acid extraction system, such as the NUCLISENS EASYMAG bench top automated nucleic acid extraction system (Roche, Inc., Indianapolis, Ind.). In one example, pelleted bacterial cells isolated from a whole blood sample can be resuspended in about 180 μl of MAGNA Pure bacteria lysis buffer and about 20 μl of Proteinase K, and then incubated at about 65° for at least about two hours. The suspension can then be heated at about 100° for about 10 minutes. The suspension can then be added to NUCLISENS EASYMAG lysis buffer (bioMérieux, Durham, N.C.) and extracted on an EASYMAG instrument (per the manufacturer's protocol), resulting in a final extraction solution having a volume of about 50 μL. An extraction solution that includes the extracted bacterial nucleic acids can then be used in subsequent procedural steps to detect and identify a bacterial infection. In some instances, the aforementioned steps allow for a high sensitivity and specificity in the subsequent diagnostic steps since the ratio of bacterial and/or fungal nucleic acids to blood cellular nucleic acids is significantly increased.

At Step 50, the presence of bacterial and/or fungal nucleic acids can be detected. There are a number of conventional techniques whereby small amounts of an isolated bacterial and/or fungal nucleic acid (e.g., bacterial and/or fungal DNA) can be amplified and then detected. Such methods are highly specific and very sensitive so as to provide optimal diagnostic value. In some instances, at least one portion of the bacterial and/or fungal nucleic acids can be selectively amplified following extraction of bacterial and/or fungal nucleic acids from bacterial and/or fungal cells. Advantageously, conventional detection methods can be used to indicate the presence of a bacterial and/or fungal infection in a single detection assay (e.g., PCR). Thus, a single amplification may be sufficient to determine whether a subject has bacteremia and/or fungemia and, if so, which bacterial and/or fungal species is responsible.

In one example, the portion of a bacterial nucleic acid amplified by the method 10 can include a mycobacterial nucleic acid sequence. A portion of bacterial nucleic acids sought to be detected can include at least a portion of a bacterial gene that encodes a mycobacterial 16S rRNA. Amplification can be carried out by any desired method, such as PCR, cloning, DNA-dependent DNA-polymerases, etc. In some instances, amplification can be carried out using real-time PCR (RT-PCR). For example, amplification can be carried out according to a modified version of the technique disclosed by Schluger, N W et al., Lancet 344:232-3 (1994). As discussed in the Example below, such modifications can include: employing asymmetric PCR by increasing the reverse primer from 0.25 μM to 0.5 μM; increasing PCR cycles from 45 to 55; and selecting step mode for melt-curve analysis.

Following amplification, the presence of bacterial and/or fungal nucleic acids in the extraction solution can be detected by, for example, gel electrophoresis, optical density, staining with markers and/or probes that specifically bind to a nucleic acid, and/or the use of melt-curve analysis. A probe specific for a particular nucleic acid may be hybridized to the nucleic acid and detected using any known technique, such as traditional Southern blotting, Northern blotting, or homogeneous protection assay.

In another aspect, detection of bacterial and/or fungal nucleic acids in the extraction solution can indicate the presence of bacteremia and/or fungemia in the subject. Conversely, failure to detect bacterial and/or fungal nucleic acid in the extraction solution may indicate that the subject is free from bacteremia and/or fungemia. In some instances, one, two, three, four or more replicates of each extraction solution can be similarly analyzed. In other instances, diagnostic results can be obtained on the same day that the blood sample is obtained from a subject.

In one example, the presence and identity of the bacterial and/or fungal species can be confirmed using post-amplification melt-curve analysis by comparison to a positive control (e.g., M. tuberculosis). In some instances, positive and negative PCR control samples can include those obtained from M. tuberculosis ATCC 27294 and PCR-grade water, respectively.

Kits

Another aspect of the present disclosure can include a kit for diagnosing or detecting bacteremia and/or fungemia in a subject. One skilled in the art will recognize components of kits suitable for carrying out a method (or methods) of the present disclosure. For example, a kit can include one or more carriers, each of which is suited for containing one or more container means, and instructions for carrying out one or more of the methods described herein. In some instances, container means can include vials, tubes, bottles, dispensers, and the like, capable of holding one or more reagents needed to practice the present disclosure. In view of the description provided herein of the present disclosure, those of skill in the art can readily determine the apportionment of the necessary reagent(s) among the container mean(s).

Instructions for kits of the present disclosure can be affixed to packaging material or can be included as a package insert. While the instructions are typically written or printed materials, they are not limited to such. Any medium capable of storing such instructions and communicating them to an end user is contemplated by the present disclosure. Such media include, but are not limited to, electronic storage media (e.g., magnetic discs, tapes, cartridges, chips), optical media (e.g., CD ROM), and the like. As used herein, the term “instructions” can include the address of an Internet site that provides the instructions. For instance, kits of the present disclosure may comprise one or more computer programs that may be used in practicing the methods of the present disclosure. For example, a computer program may be provided that takes the output from microplate reader or realtime-PCR gels or readouts and prepares a calibration curve from the optical density observed in the wells, capillaries or gels, and compares these densitometric or other quantitative readings to the optical density or other quantitative readings in wells, capillaries, or gels with test samples.

In one example, a kit can include reagents and instructions for carrying out the method 10 described above. In some instances, the kit can include a first container means for holding a lysing agent and a second container means holding one or detection agents (e.g., oligonucleotide primers, probes, etc.) for detecting bacterial and/or fungal nucleic acids, as well as reagents associated therewith (e.g., polymerases, dNTPs, PCR-grade water, etc.). In other instances, the kit can include additional carrier means holding solutions needed for collection and storage of blood (e.g., whole blood), a carrier means holding washing or cleaning solutions (e.g., PBS), and/or carrier means holding a bacterial and/or fungal lysing agent. In further instances, the kit can include carrier means (e.g., vials, tubes, etc.) for holding blood obtained from a subject. In still further instances, the kit can include instruments for obtaining a blood sample from a subject (e.g., a hypodermic needle and syringe). In other instances, the kit can include one or more carrier means holding reagents for detecting bacterial and/or fungal nucleic acids, such as electrophoresis gels.

In addition to the clinical uses discussed herein, kits of the present disclosure can be used for experimental applications.

The following example is for the purpose of illustration only and is not intended to limit the scope of the claims, which are appended hereto.

### Example

In order to investigate the performance of a nucleic acid amplification test (NAAT) following large volume extraction of whole blood for the diagnosis of M. tuberculosis bacteremia compared with mycobacterial blood culture, we studied consecutive febrile admissions to two hospitals in Tanzania, a country experiencing a generalized HIV epidemic and high incidence of tuberculosis.

### Materials and Methods

### Blood Culture and Collection

Samples for blood cultures, NAAT, and other diagnostic tests were collected from patients aged ≧13 years hospitalized at the Kilimanjaro Christian Medical Centre (KCMC) and Mawenzi Regional Hospital (MRH) in Moshi, Tanzania, from July 2006 through October 2009. Patients with oral temperatures≧38.0° C. were invited to participate in the study. For qualifying study participants 5 mL of blood was inoculated into a BacT/ALERT mycobacterial (MB) bottle (bioMérieux, Durham, N.C.) and 5 mL into an EDTA tube for subsequently nucleic acid amplification. For study participants with oral temperature≧38.0° C., HIV-infection, fever of >1 month duration, and presumed or measured weight loss>10%, blood was also inoculated into a BACTEC MYCO/F-Lytic bottle and Wampole ISOLATOR 10 lysis-centrifugation tube. All participants received HIV counseling and testing.

### Adequacy of Blood Volume

Blood culture bottles and tubes were assessed for volume adequacy by comparing the weight before and after inoculation with blood. A bottle or tube was considered adequately filled if it contained 4 to 6 mL of blood. Only samples from patients with adequately filled bottles and tubes were included in the study.

### Processing of Blood Cultures

After assessment of the adequacy of the blood volume, BacT/ALERT MB bottles were loaded into the BacT/ALERT 3D automated microbial detection system (BioMérieux Inc., Durham, N.C.) where they were incubated for 42 days. BACTEC MYCO/F-Lytic bottles (Becton Dickinson, Franklin Lakes, N.J.) were incubated at 35° C. for 42 days; bottle bottoms were examined for fluorescence daily using Wood's lamp. ISOLATOR10 lysis-centrifugation tubes were centrifuged and processed using the Wampole ISOSTAT/ISOLATOR Microbial System (Inverness Medical, Princeton, N.J.), plated to Middlebrook 7H10 agar, and incubated at 35° C. in 5% CO₂ for 42 days. An aliquot of the blood-broth mixture was removed from bottles flagged positive by the instrument or by inspection of the bottom with a 5 mL syringe and 19 g needle for MB and MYCO/F-Lytic. A portion was examined by Kinyoun stain (MB and MycoF-Lytic), Gram stain, and India ink stain when yeasts were observed. Aliquots were plated to solid media according to stain results. Mycobacterial plates were examined weekly for growth. AccuProbe Culture Identification Test MTB and MAC kits (Gen-Probe Inc., San Diego, Calif.) were used to identify members of M. tuberculosis complex and MAC. Mycobacteria other than M. tuberculosis and MAC were identified at the National Institute for Public Health and the Environment, Bilthoven, the Netherlands using techniques described elsewhere. Colonies growing on Middlebrook 7H10 plates from lysis-centrifugation specimens were counted and colony forming units (CFU) per mL of blood were calculated. Negative companion bottles from positive sets were sub-cultured at the end of the 42-day protocol. Identification of growth was performed by standard techniques.

### Classification of Case and Control Specimens

Specimens were classified as being from a case patient with M. tuberculosis bacteremia if the MB blood culture bottle was positive for M. tuberculosis. Those with mycobacterial blood cultures negative for M. tuberculosis were classified as controls. Controls were selected to include patients with and without HIV infection; with bloodstream infections due to mycobacteria other than M. tuberculosis; with non-mycobacterial bloodstream infections; and with negative blood cultures. The results of clinical evaluations and examination of non-blood specimens for mycobacteria were not considered in the designation of cases and controls, but were available for evaluation following completion of nucleic acid amplification testing.

### Processing of EDTA Blood

EDTA blood was transferred to cryovials and stored at −80° C. Once sample collection was completed, cryovials containing whole blood were shipped on dry ice to the Cleveland Clinic for nucleic acid amplification testing.

### DNA Extraction

Whole blood samples were received frozen and maintained at −70° C. until processed. Each 5 mL sample was thawed, mixed thoroughly, and transferred into an adult ISOLATOR tube (Wampole Laboratories, Cranbury N.J.). Each Isolator tube was gently vortexed for 5-10 seconds and held at room temperature for at least one hour to inactivate HIV, if present. Following centrifugation at 4,700 rpm for 30 minutes, a pellet was obtained using the manufacturer's instructions. The 1.5 mL pellet was transferred into a 2 mL Sarstedt microcentrifuge tube and centrifuged at 9400 rpm for 10 minutes. Approximately 1.2 mL of supernatant was removed and 500 μL Phosphate Buffered Saline (PBS) was added. The suspension was vortexed, centrifuged at 9400 rpm for 10 minutes, and most of the supernatant was removed. One hundred and eighty microliters of MAGNA PURE bacteria lysis buffer (Roche, Indianapolis Ind.) and 20 μL Proteinase K (Roche) was added to each pellet and incubated at 65° C. for at least 2 hours to overnight. The suspension was heated at 100° C. for 10 minutes. Processing of the pellet was performed using a biosafety 2 hood and a microcentrifuge with a removable rotor. The entire sample was added to 2 mL of NUCLISENS EASYMAG lysis buffer (bioMérieux, Durham, N.C.) and extracted on the EASYMAG instrument. A final extraction volume of 50 μL was obtained.

### Real-Time PCR

PCR was performed using the LightCycler system (Roche) based on a previously described assay, with the following modifications. Asymmetric PCR was used by increasing the reverse primer from 0.25 μM to 0.5 μM. Additionally, PCR cycles were increased from 45 to 55 and step mode was selected for melting curve analysis. Positive and negative controls consisted of M. tuberculosis ATCC 27294 and PCR grade water respectively. If amplification occurred, then the identity of the Mycobacterium species as M. tuberculosis was confirmed using post-amplification melt curve analysis by comparison to the positive control (±2° C.). Five replicates of each sample were tested.

### Data Analysis

Means and ranges were calculated for continuous data and compared by the paired two-sample t test for means after log transformation was performed to correct for the observed positively skewed (nonparametric) distributions. Proportions were compared using the chi-square test with Yates' correction for small numbers when necessary. Sensitivity, specificity, positive predictive value, and negative predictive values were calculated for the NAAT compared with blood culture. All analyses were done with the SAS system for Windows (release 9.1; SAS Institute, Cary, N.C.).

### Research Ethics

This study was approved by the KCMC Research Ethics Committee, the Tanzania National Institutes for Medical Research National Research Ethics Coordinating Committee, and an Institutional Review Board of Duke University Medical Center.

### Results

Of 91 participants included in the study, 25 (27.5%) had M. tuberculosis bacteremia and were classified as cases. All were HIV-infected. The remaining 66 (72.5%) had mycobacterial blood cultures negative for M. tuberculosis and were classified as controls. Controls included 2 (3.0%) participants with bloodstream infections due to mycobacteria other than M. tuberculosis; 5 (7.6%) with Cryptococcus neoformans; 7 (10.6%) with Escherichia coli; 3 (4.5%) with Streptococcus pneumoniae; 16 (24.2%) with Salmonella enterica serotype Typhi; and 33 (50.0%) with negative blood cultures. Twenty four (36.4%) control patients were HIV-infected.

| Designation | HIV serostatus | Invasive infection category | Bloodstream isolate | N (%) |
|---|---|---|---|---|
| Case | Infected | Met study eligibity; | M. tuberculosis | 25 (27.5) |
| | | M. tuberculosis bloodstream | | |
| | | infection | | |
| Control | Infected | Nontuberculous | M. sherrisii (1), | 2 (2.2 |
| | | mycobacterial bloodstream | M. simiae (1) | |
| | | infection | | |
| | Infected | Met study eligibility, blood | Negative | 13 (14.3) |
| | | culture negative | | |
| | Infected | Non-mycobacterial | C. neoformans (5), | 9 (9.9) |
| | | bloodstream infection | E. coli (3), | |
| | | | S. pneumoniae (1) | |
| | Unifected | Non-mycobacterial | E. coli (4), | 22 (24.2) |
| | | bloodstream infection | S. pneumonia (2), | |
| | | | Salmonella Typhi (16) | |
| | Unifected | Blood culture negative | Negative | 20 (22.0) |

| | |
|---|---|
| Total | 91 (100) |

Patient samples with and without M. tuberculosis bacteremia selected for evaluation of nucleic acid amplification test.

Of 25 samples with M. tuberculosis bacteremia, 9 (36.0%) were positive by nucleic acid amplification test (NAAT). Of those positive by NAAT, the mean (range) number of replicates positive was 3 (1, 5). For those with results available, the mean (range) magnitude of mycobacteremia was 58.1 (17.0, 90.0) CFU/mL among NAAT positive samples compared with 0.5 (0.5, 0.5) CFU/mL for NAAT negative samples (p=0.157). The mean (range) time to positive in the continuously monitored BacT/ALERT MB system was 16.8 (9.4, 27.5) days for NAAT positive samples and was 22.0 (11.3, 30.9) days for NAAT negative samples (p=0.062).

| | M. tuberculosis | Replicates | | MB time to |
|---|---|---|---|---|
| | nucleic acid | positive | M. tuberculosis | positive |
| Sample | result | n (%) | CFU/mL | (days) |
| 1 | − | — | ND | 19.2 |
| 2 | − | — | ND | 11.3 |
| 3 | + | 3 (60.0) | 78.3 | 16.7 |
| 4 | + | 3 (60.0) | 47.2 | 20.2 |
| 5 | − | — | ND | 30.9 |
| 6 | − | — | ND | 26.2 |
| 7 | + | 5 (100.0) | ND | 13.8 |
| 8 | − | — | ND | NA |
| 9 | − | — | ND | NA |
| 10 | − | — | Negative | NA |
| 11 | − | — | Negative | 21.0 |
| 12 | + | 3 (60.0) | 90.0 | 9.4 |
| 13 | − | — | Negative | NA |
| 14 | − | — | Negative | 29.0 |
| 15 | + | 1 (20.0) | ND | 18.8 |
| 16 | − | — | Negative | NA |
| 17 | − | — | 0.5 | 20.0 |
| 18 | − | — | ND | 24.8 |
| 19 | − | — | Negative | 15.3 |
| 20 | − | — | ND | 22.1 |
| 21 | + | 1 (20.0) | ND | 17.2 |
| 22 | − | — | Negative | NA |
| 23 | + | 3 (60.0) | Negative | NA |
| 24 | + | 5 (100.0) | 17.0 | 10.8 |
| 25 | + | 1 (20.0) | Negative | 27.5 |

ND: Not done; NA: Not available

Of 66 control samples, 1 (1.5%) was positive for M. tuberculosis by NAAT. The sample was positive in 1 of 5 replicates. Evaluation of case report forms showed that this participant was a 37 year old admitted with fever, rigors, cough, hemoptysis, and dyspnea. The patient was tachycardic, tacypneic, hypotensive, and hypoxic, with a BMI of 24.1. Chest examination revealed crepitations, bronchial breathing, and a pleural rub. Hepatomegaly was present. The patient was diagnosed clinically with pneumonia. HIV antibody testing was negative. Chest radiograph and sputum studies for M. tuberculosis were not done.

The sensitivity (95% CI) of the NAAT for the diagnosis of M. tuberculosis bacteremia was 0.360 (0.187, 0.573) and the specificity was 0.985 (0.907, 0.999). The positive predictive value (95% CI) of the NAAT for the diagnosis of M. tuberculosis bacteremia was 0.90 (0.541, 0.995) and the negative predictive was 0.802 (0.696, 0.879).

We demonstrate that extraction of 5 mL of whole blood followed by RT-PCR targeting the mycobacterial 16S rRNA gene detected approximately one third of patients with M. tuberculosis bacteremia diagnosed by culture of the equivalent volume of blood. Specificity exceeded 98% in a control population that included HIV-infected persons enrolled in a country with a high incidence of tuberculosis. There was a trend towards patients with higher magnitude of mycobacteremia being more likely to have a positive M. tuberculosis NAAT test. The specificity of our assay was high and exceeded that observed in PCR studies of whole blood specimens for the diagnosis of pulmonary tuberculosis.

From the above description, those skilled in the art will perceive improvements, changes and modifications. Such improvements, changes, and modifications are within the skill of one in the art and are intended to be covered by the appended claims. All references cited herein and listed above are incorporated by reference in their entireties as needed and as discussed herein.

Having described the invention, the following is claimed:

1. A method of diagnosing bacteremia and/or fungemia in a subject, the method comprising:
obtaining a blood sample from the subject;
contacting the blood sample with a lysing agent under conditions in which both red and white blood cells are lysed in the blood sample and bacterial and/or fungal cells remain intact;
extracting bacterial and/or fungal nucleic acids from bacterial and/or fungal cells in the blood sample, respectively; and
detecting the presence of bacterial and/or fungal nucleic acids in the blood sample, wherein the presence of bacterial and/or fungal nucleic acids in the blood sample is indicative of the subject having bacteremia and/or fungemia, respectively.

2. The method of claim 1, the lysing agent comprising a saponin.

3. The method of claim 1, the bacteremia comprising Mycobacterium tuberculosis bacteremia.

4. The method of claim 1, the step of extracting bacterial and/or fungal nucleic acids from the blood sample further comprising:
contacting the blood sample with a bacterial and/or fungal lysing agent to release the bacterial and/or fungal nucleic acids sought to be detected; and
isolating the bacterial and/or fungal nucleic acids.

5. The method of claim 1, further comprising the step of amplifying at least one portion of the bacterial and/or fungal nucleic acids in the blood sample.

6. The method of claim 5, the step of amplifying at least one portion of the bacterial and/or fungal nucleic acids in the blood sample further comprising performing real-time polymerase chain reaction (RT-PCR) to target at least one segment of a bacterial gene that encodes a mycobacterial 16S rRNA.

7. The method of claim 1, the step of detecting the presence of bacterial and/or fungal nucleic acids from the blood sample further comprising performing post-amplification melt-curve analysis.

8. The method of claim 1, wherein the blood sample obtained from a subject is about 1 ml to about 40 ml in volume.

9. The method of claim 1, wherein the blood sample obtained from a subject is about 4 ml to about 6 ml in volume.

10. The method of claim 1, the blood sample obtained from the subject being whole blood.

11. A method of diagnosing Mycobacterium tuberculosis bacteremia in a subject, the method comprising:
obtaining a blood sample from the subject;
contacting the blood sample with a lysing agent under conditions in which both red and white blood cells are lysed in the blood sample and Mycobacterium tuberculosis cells remain intact;
extracting Mycobacterium tuberculosis nucleic acids from Mycobacterium tuberculosis cells in the blood sample; and
detecting the presence of Mycobacterium tuberculosis nucleic acids in the blood sample, wherein the presence of Mycobacterium tuberculosis nucleic acids in the blood sample is indicative of the subject having Mycobacterium tuberculosis bacteremia.

12. The method of claim 11, the lysing agent comprising a saponin.

13. The method of claim 11, the step of extracting Mycobacterium tuberculosis nucleic acids from the blood sample comprising:
contacting the blood sample with a Mycobacterium tuberculosis lysing agent to release the bacterial nucleic acids sought to be detected; and
isolating the Mycobacterium tuberculosis nucleic acids.

14. The method of claim 11, further comprising the step of amplifying at least one portion of the Mycobacterium tuberculosis nucleic acids in the blood sample.

15. The method of claim 14, the step of amplifying at least one portion of the Mycobacterium tuberculosis nucleic acids in the blood sample comprising performing RT-PCR to target at least one portion of a bacterial gene that encodes a mycobacterial 16S rRNA.

16. The method of claim 11, the step of detecting the presence of Mycobacterium tuberculosis nucleic acids from the blood sample further comprising post-amplification melt-curve analysis.

17. The method of claim 11, wherein the blood sample obtained from a subject is about 1 ml to about 40 ml in volume.

18. The method of claim 11, wherein the blood sample obtained from a subject is about 4 ml to about 6 ml in volume.

19. The method of claim 11, the blood sample obtained from the subject being whole blood.

20. A kit for performing the method of claim 1.
